SEQUENCE LISTING (23 sequences)

SEQ ID NO: 1 - DNA, 25 bp, Artificial Sequence
  forward primer unmethylated promoter; target sequence FMR1
gtgtttgattgaggttgaatttttg

SEQ ID NO: 2 - DNA, 28 bp, Artificial Sequence
  backward primer unmethylated promoter; target sequence FMR1
atttaatttcccacrccactaaatacac

SEQ ID NO: 3 - DNA, 27 bp, Artificial Sequence
  forward primer methylated promoter; target sequence FMR1
gttgcgggtgtaaatattgaaattacg

SEQ ID NO: 4 - DNA, 26 bp, Artificial Sequence
  forward primer methylated promoter; target sequence XIST
aattaaagtaggtattcgcggtttcg

SEQ ID NO: 5 - DNA, 26 bp, Artificial Sequence
  backward primer methylated promoter; target sequence XIST
tttttccttaacccatcgaaatatcg

SEQ ID NO: 6 - DNA, 27 bp, Artificial Sequence
  forward primer unmethylated promoter; target sequence XIST
aaaagtggttgttattttagatttgtt

SEQ ID NO: 7 - DNA, 25 bp, Artificial Sequence
  backward primer unmethylated promoter; target sequence XIST
ctacctcccaatacaacaatcacac

SEQ ID NO: 8 - DNA, 29 bp, Artificial Sequence
  forward primer unmethylated repeat; target sequence FMR1
tttgagaggtgggttgtgggtgtttgagg

SEQ ID NO: 9 - DNA, 34 bp, Artificial Sequence
  backward primer unmethylated repeat; target sequence FMR1
aacaccactaccaaaaaacatacaacaacacaac

SEQ ID NO: 10 - DNA, 30 bp, Artificial Sequence
  forward primer methylated repeat; target sequence FMR1
ccgcctctaaacgaacgacgaaccgacgac

SEQ ID NO: 11 - DNA, 29 bp, Artificial Sequence
  backward primer methylated repeat; target sequence FMR1
tttcgagaggtgggttgcgggcgttcgag

SEQ ID NO: 12 - DNA, 29 bp, Artificial Sequence
  forward primer methylated promoter; target sequence FMR1
cgtcgtcggttcgtcgttcgtttagaggc

SEQ ID NO: 13 - DNA, 28 bp, Artificial Sequence
  backward primer methylated promoter; target sequence FMR1
ccgaccgattcccaacaacgcgcatacg

SEQ ID NO: 14 - DNA, 21 bp, Artificial Sequence
  primer used to detect and evaluate in a single reaction the occurrence and extent of a possible aberrant methylation of promoter region FRAX E-A
ttcgtcgtcgttgtcgtcgtc

SEQ ID NO: 15 - DNA, 27 bp, Artificial Sequence
  primer used to detect and evaluate in a single reaction the occurrence and extent of a possible aberrant methylation of promoter region FRAX E-B
aactaaaaatatccgaaccgcatcgac

SEQ ID NO: 16 - DNA, 21 bp, Artificial Sequence
  primer used to detect and evaluate in a single reaction the occurrence and extent of a possible aberrant methylation of promoter region FRAX F-A
agttcgtagcgcggattttcg

SEQ ID NO: 17 - DNA, 25 bp, Artificial Sequence
  primer used to detect and evaluate in a single reaction the occurrence and extent of a possible aberrant methylation of promoter region FRAX F-B
aacgtaaacgcgactaacgctaacg

SEQ ID NO: 18 - DNA, 22 bp, Artificial Sequence
  primer used to detect a skewed X inactivation [hum-A2(m)]
aatttgttttagagtgtgtgtg

SEQ ID NO: 19 - DNA, 21 bp, Artificial Sequence
  primer used to detect a skewed X inactivation [hum-B2(m)]
gcgagcgtagtatttttcggc

SEQ ID NO: 20 - DNA, 21 bp, Artificial Sequence
  primer used to detect a skewed X inactivation [hum-C(com)]
ctactacctaaaactaatctc

SEQ ID NO: 21 - DNA, 27 bp, Artificial Sequence
  primer FX-K; target methylated FMR1
ggaagtgaaatcgaaacggagttgagc

SEQ ID NO: 22 - DNA, 28 bp, Artificial Sequence
  primer FX-J; target methylated FMR1
aacgttctaaccctcgcgaaacaatacg

SEQ ID NO: 23 - DNA, 57 bp, Artificial Sequence
  expansion of a sequence found in FraX-F- positive patients; normal persons (GCCGTC)n(GCC)m, m=12 to 26 and n=3
gccgtcgccgtcgccgtcgccgccgccgccgccgccgccgccgccgccgccgccgcc

(19) United States
(12) Patent Application Publication
(10) Pub. No.: US 2003/0170649 A1
(43) Pub. Date: Sep. 11, 2003

(54) Method for detecting and evaluating a potentially aberrantly methylated dna region on the x chromosome, or the clonality

(76) Inventors: Oskar A. Haas, Vienna (AT); Andreas Weinhausel, Vienna (AT)

Correspondence Address: BIRCH STEWART KOLASCH & BIRCH, PO BOX 747, FALLS CHURCH, VA, 22040-0747, US

(21) Appl. No.: 10/148,570

(22) PCT Filed: Dec. 4, 2000
(86) PCT No.: PCT/AT00/00324

(51) Int. Cl. C12Q 1/68; C12P 19/34

(52) U.S. Cl. 435/6; 435/91.2

(57) ABSTRACT

A method for detecting and evaluating a potentially aberrantly methylated DNA region on the X chromosome, or the clonality, wherein the extent of methylation of the potentially aberrantly methylated DNA region and optionally the extent of methylation in a definitely physiologically methylated DNA region of the X chromosome and/or the extent of methylation as well as optionally the sequence variant of a polymorphous DNA region of the X chromosome, which may either be physiologically or aberrantly methylated, are determined in a sample and the presence and optionally the variant and the extent of the potential aberration, or the clonality, are subsequently diagnosed from the comparison of the methylation determinations and optionally the sequence variant.

There is provided a method for detecting and evaluating a potentially aberrantly methylated DNS region on the X chromosome, or the clonality, the use of such a method as well as a kit for carrying out said method.

DNA, the carrier of genetic information, is present in the nuclei of eukaryotes tightly packed in structural units, the chromosomes. Changes in the numbers or even structures of the chromosomes as well as alterations of the DNA itself without inducing microscopic alterations on the chromosomes usually result in serious syndromes in men. Diseases can also be caused by epigenetic alterations such as by an aberrant DNA methylation. DNA methylation occurs by the covalent binding of methyl groups to nucleotides and constitutes an important regulation mechanism: In the case of the mammalian genome, active genes or their regulatory units are frequently undermethylated, while inactive DNA sections are frequently characterized by strong methylation. Therein, primarily the cytosine residues in CpG dinucleotides are methylated in 5′. Accordingly, in methylated DNA, methyl cytosine (mC) is preferably found as mCpG in the CpG islets of DNA. In most cases, these are to be found in regulatory DNA units and originally unmethylated. Physiologically, unmethylated active DNA and methylated inactive DNA sections are usually found in the diploid mammalian genome mCpG in imprinted genes—as a function of the parental origin of the respective gene or chromosome. Thus, imprinted genes are expressed by just one of the two homologous chromosomes. A similar ratio is found in cells of female individuals for the dose compensation of genes that are localized on X chromosomes: since female cells (XX), unlike male cells (XY), carry two X chromosomes, one of the two X chromosomes is inactivated. This inactive X chromosome, or its DNA (in the form of mCpG), is methylated except for a few regions (XIC . . . X inactivation center, pseudo-autosomal regions). This X inactivation occurs randomly in the course of the development of an individual (random X inactivation), whereupon the entirety of cells consists of a population of cells with inactive paternal and active maternal X chromosomes as well as an equally large population of cells with active paternal and inactive maternal X chromosomes. Hence results a ratio of active to inactive (Xa/Xi) alleles of Xa/Xi=50/50 in respect to their parental origin. In many cases, also a deviation from this 50/50 ratio may, however, occur in female individuals such that, although the ratio of methylated to unmethylated, or active to inactive, X chromosomes (per cell and also per overall population) of 1:1 is maintained, the size of the cell population with the active/inactive chromosome does not equal 50/50 in respect to the parental origin of the same. This means that the cell population (clone) with the active X chromosome of one parent (E1) is larger and that with the active X chromosome of the other parent (E2) is smaller, or the clone with the inactive X chromosome of E1 is smaller and that with the inactive X chromosome of E2 is larger. This phenomenon of a “shifted” clone size or clonality is described by the term “skewed X inactivation”, which can be observed even in normal females with increasing age.

Experimentally, the methylation of DNA can be detected, for instance, by cutting with methylation-sensitive enzymes, by means of mC-specific antibodies, by genomically sequencing bisulfite-deaminated DNA, by the selective hydrazine cleavage of unmethylated DNA, etc.

If a DNA section comprises an incorrect (aberrant) methylation (i.e. a hypermethylation of physiologically unmethylated DNA or a hypomethylation of physiologically methylated DNA), this will in most cases lead to a deregulation of the physiological function of this DNA section and can result in a disease. The determination of the phenotype is frequently insufficient to diagnose a particular disease. In order to be able to determine the disease for sure and to diagnose also the variant or disease, an analysis of the hereditary substance of the patient, in particular the methylation of a given DNA section, are usually required.

A number of diseases are caused by an alteration of the DNA on an X chromosome. A DNA region may, for instance, be methylated on the X chromosome so as to inactivate this gene. In addition, the gene function can be disturbed and additionally methylated, for instance, by an alteration (mutation) of the DNA, for instance by the expansion of a repeat region, (FraX-A, FraX-E, FraX-F) (cf. Carrel et al., American Journal of Medical Genetics 64:27-30 (1996); Hirst et al., Hum. Molec. Genet. 2: 197-200, 1993; Parrish et al., Nature Genet. 8 : 229-235, 1994 ; Ritchie et al., Hum. Molec. Genet. 3 : 2115-2121, 1994 ; Sutherland et al., Hum. Molec. Gent. 1 : 111-113, 1992). In other cases, diseases may go back to alterations of DNA sections or whole chromosomes (regions) by duplication, insertion, translocation. If such an alteration affects the X chromosome already in the early development stages of an individual, one will have to speak of X-chromosomal diseases in many cases. Also with sporadic diseases, as is the case with many tumor diseases, X-chromosomal alterations could be proved (Gale et al., Blood, Vol. 83, No. 10 (1994), 2899-2905). Consequently, different variants of the X chromosome are present concurrently, or also different cell populations (clones) besides the normal, healthy clone. These populations (clones) may differentiate both in the allele pattern and in the methylation pattern of the alleles or the altered genetic material, respectively (cf. Cammarata et al., Am. J. Med. Genet., 1999; 85(1): 86-7; ISSN: 0148-7299; Dierlamm et al., Br. J. Haematol., 1995; 91 (4): 885-91; ISSN: 0007-1048; James et al., Ann. Hum. Genet., 1997; 61(6): 485-90; ISSN: 0003-4800; Leal et al., Hum. Genet., 1994; 94(4): 423-6; ISSN: 0340-6717; MacDonald et al., Hum. Mol. Genet., 19,94; 3(8): 1365-71; ISSN: 0964-6906; und Martinez-Pasarell et al., Horm. Res., October 1999; 51(5): 248-252; ISSN: 0301-0163).

In genetic assays the affected gene, or the affected gene locus, is assayed by cytogenetic, Southern blot, PCR, reverse transcription PCR (RT-PCR) or immunologic analyses. Those assays serve to analyze the sequence, the expression, the extent of methylation and the like.

The fragile X syndrome (FX or FraX-A syndrome) is an example of a hereditary disease which affects primarily males. The clinical phenotype of the FX syndrome displays moderate to severe mental retardation, large ears, a long face, hyperactivity, autistic traits, etc. The FX syndrome results from a functional failure or a suppressed expression of the FMR 1 gene, which is located in the telomeric region of the long arm of the X chromosome, X (q27). In the vast majority of cases, the gene transcription is impaired by an unphysiologic expansion of a polymorphous CGG-triplet repeat region on the 5′ end of the FMR 1 gene. In normal individuals, the FMR 1 gene repeat region comprises between 6 and (48-) 54 repeat units with an occurrence maximum of the allele frequency having a repeat length of approximately 30 units. Individuals with alleles in the so-called premutation comprise between (48-) 54 and 200 repeat units and do not show any clinical failures, either. There is a grey zone in the range of between 48 and 54 repeat units, i.e., patients having a repeat region length of 48-54 units will have to be classified as premutations with further FX patients in the family. Individuals with premutations have increased risks to pass on full mutations to the subsequent generation, such full mutations consisting of more than 200 triplet repeat units. The risk to transmit full mutations to subsequent generations depends on the size of the premutation, on the sex of the transmitting parent and on the position of the parent in the family tree. Individuals with larger premutation alleles bear higher risks to transmit full mutations than those having shorter premutations. However, this phenomenon only relates to females such that children from mothers with premutations can be afflicted by full mutations. On the other hand, children from fathers having a premutation allele will only inherit the premutation.

Moreover, other rare mutations such as, e.g., small deletions of the FMR 1 gene region or point mutations within the coding sequence, can induce a lack of, or the production of a functionally incapable, FMR 1 protein.

It could be demonstrated that the FMR 1 gene region with a normal or premutation repeat number is not methylated, while, in the event of full mutations, cytosines in CpG dinucleotides are methylated in the expanded repeat region and in the surrounding 5′ promoter region of the FMR 1 gene.

Nevertheless, FMR 1 alleles with a vast number of repeat units (300-800) can be reactivated in vitro by treatment with demethylating agents such as 5-azadeoxycytidine. In addition, males were found who, despite repeat numbers of above 200 (full mutation), which were, however, unmethylated, showed normal intelligence and almost normal FMR 1 protein concentrations. From this, it can be concluded that the de novo methylation of the FMR 1 promoter is more likely to impair the gene function than the length of the expanded repeat region. Different degrees of methylation can result in different degrees of the disease.

Current methods for evaluating the FX syndrome comprise cytogenetic, Southern blot, PCR, reverse transcription PCR (RT-PCR) and immunohistochemical analyses. These assays, as a rule, analyze the size of the expanded repeat region, the de novo methylation of this gene region or the gene expression.

In WO 92/14840, the detection of the fragile X syndrome by the aid of restriction enzymes is described, wherein the restriction enzymes, for instance, cut only those sites which include unmethylated cytosines. Unlike unmethylated DNA, methylated DNA is, thus, digested and the digestion products are detectable.

WO 91/09140 relates to an oligonucleotide having a defined sequence and binding to a given site, M54, of the region of the fragile gene. Patients with fragile X syndrome can be detected by in situ hybridization with the labeled oligonucleotide.

WO 92/20825 likewise relates to a method for diagnosing the fragile X syndrome, wherein either the amount of mDNA is determined, for instance, by RT-PCR or the amount of protein is determined, for instance, by means of immunological methods of the FMR 1 gene. Another method for detecting the fragile X syndrome consists in determining the length of the repeat region. This is done, for instance, by the digestion of the FMR 1 gene with restrictions enzymes or by PCR methods with primers that are specific for the repeat region of the FMR 1 gene, and subsequent gel electrophoresis.

Also the method according to U.S. Pat. No. 5,658,764 for detecting the fragile X syndrome comprises size measurements of the GC-rich sequence by PCR methods.

The drawbacks of the above-described methods consist in that the analysis of but one gene region does not allow an unambiguous assertion as to the variant of the disease. The conventional PCR of a single gene region, for instance, does not yield sufficiently clear results, because in the case of the fragile X syndrome full mutations can, for instance, not be amplified by PCR on account of their high CG content and their repetitive nature. Since negative results might, thus, also suggest full mutations, a Southern blot assay would have to be carried out subsequently. Such Southern blot assays yield information on the length of the repeat region as well as on the methylation. The drawbacks of Southern blotting reside in that it is time-consuming, that relatively large quantities of DNA are necessary, which involves difficulties particularly in prenatal analyses, and also the usually required use of radioisotopes is disadvantageous to the laboratory personnel. A disadvantage of PCR consists in that it cannot distinguish between a full mutation and a premutation, in particular in the event of a borderline full mutation/premutation, i.e., the length of the repeat region is about 200 units and the sequence is partially methylated and partially unmethylated. Furthermore, the distinction between a full mutation and a mosaic (i.e., e.g., a full mutation on one allele and a premutation or an expansion in the normal range on the other allele) is problematic or impossible. Moreover, those known methods are very time-consuming, requiring several method steps. In females, those methods also involve high “false-negative” rates.

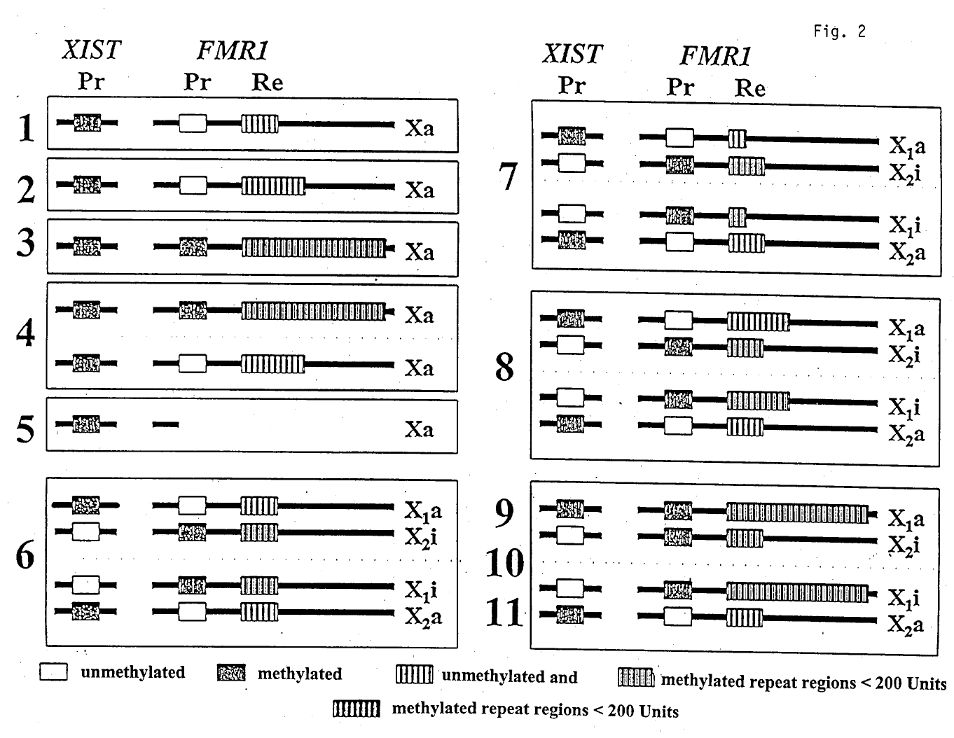

The publication of Das et al. “Methylation Analysis of the Fragile X Syndrome by PCR” (Genetic Testing, Vol. 1, No. 3, 1997/98) relates to a method for detecting the fragile X snydrom by methylation-specific PCR (MS-PCR). Thereby, the unmethylated cytosine residues are converted into uracil by the addition of an agent, while the methylated cytosine residues are not converted. In DNA replication, uracil is replaced with thymidine in the modified DNA. By constructing specific primers that hybridize with the modified DNA sequence, either the methylated or the unmethylated sequence is amplified. If primers resulting in varying product lengths are selected, it is feasible to determine as a function of the PCR product whether a given site of the FMR 1 gene is methylated or not. Due to the PCR amplification of the unmethylated sequence, MS-PCR distinguishes normal and premutated alleles from full mutation and mosaic sequences—amplification of the methylated sequence. After this, normal and premutated sequences must be distinguished by conventional PCR (p. 152, col. 2, 2^{nd} paragraph). In conventional PCR, only normal sequences (5-50 repeat units) but no premutated sequences (50-200 repeat units) are amplified (cf. FIG. 2B). Furthermore, MS-PCR cannot differentiate between individuals with full mutations and those having full mutation/premutation mosaics. Such a determination must be carried out subsequently by Southern blot analysis (cf. p. 153, col. 2 below to p. 154, col. 1 on top). It is, moreover, impossible to test female syndromes by said MS-PCR, because the inactive X chromosome is always methylated and hence a product will be obtained in any event. Afflicted female patients consequently can be correctly diagnosed only by conventional Southern blot analysis (cf. p. 155, last-but-one paragraph).

Even when carrying out the above-mentioned methods in combination, the determination of the clone size, i.e., the clonality, is impossible in the event of a mosaic, since in that case only methylation or non-methylation is qualitatively assessed. This can only be realized by analyzing an additional informative gene section (i.e., two distinguishable alleles), whereby the extent of a skewed X inactivation must be performed on the X chromosome by the methylation analysis of such an informative region. In addition, this region has to be chosen in a manner that the physiologic methylation pattern is known beforehand. A gene locus frequently used to this end is the HUMARA (human androgen receptor) gene (cf. Pardini et al., The New England Journal of Medicine, Vol. 338, No. 5 (1998), 291-295; Kubota et al., Hum. Gent. (1999) 104: 49-55).

These problems are faced in the diagnostics of all X-chromosomal diseases whose pertinent gene regions are differently methylated by X inactivation and, in particular, in the detection of the variant of the disease (normal mutation/premutation/full mutation) and the clonality or clone size. Examples encompass FraX-A, FraX-E, FraX-F and various X-chromosomal diseases:

As already pointed out in connection with the fragile X syndrome (FraX-A), the ratios with FraX-E and FraX-F are similar, the disease likewise developing by repeat expansion and concurrent methylation.

In FraX-E-positive individuals, a GCC repeat expansion present at Xq28 adjacent a CpG islet, comprises more than 200 GCC units as compared to 6-25 GCC units in normal individuals. Besides, the CpG islet is methylated in FraX-E-positive individuals. Those patients show mental retardations (cf. Knight et al., Am. J. Hum. Gent. 53:A79, 1993; Knight et al., Cell 74: 127-134, 1993). A gene, FMR2, which is transcribed distally from the CpG islet with FraX-E and down-regulated by repeat expansion and methylation was identified (Gu et al., Nature Gent. 13: 109-113).

An expansion of a sequence (GCCGTC)ₙ(GCC)ₘ can be found in FraX-F-positive patients, wherein m can be more than 900 and the adjacent CpG islet is methylated. In normal persons, m is 12 to 26 and n is 3 (Ritchie et al., Hum. Molec. Gent. 3: 2115-2121, 1994).

The drawbacks of the known assaying techniques reside in that several different method steps are frequently necessary to diagnose the diseases and the disease variants. In many cases, an unambiguous assertion as to the syndrome is not possible, because the applied assaying techniques only enable assertions about specific gene regions.

It is, therefore, an object of the present invention to provide a method for diagnosing a potentially aberrantly methylated DNA region on the X chromosome, or the clonality, which can be realized rapidly and readily by as few methods steps as possible with distinction being made between the various variants, and which method can be applied in both male and female patients to distinguish between different genotypes.

The method according to the invention of the initially defined kind is characterized in that

a) either the extent of methylation of the potentially aberrantly methylated DNA region and/or the extent of methylation in a definitely physiologically methylated DNA region of the X chromosome is determined in a sample and the presence and optionally the variant and the extent of the potential aberration are subsequently diagnosed from the comparison of the methylation determinations, or

b) the extent of methylation of the potentially aberrantly methylated DNA region and/or the extent of methylation as well as optionally the sequence variant of a polymorphous DNA region of the X chromosome, which may either be physiologically or aberrantly methylated, are determined in a sample, and the presence and optionally the variant and the extent of the potential aberration, or the clonality, are subsequently diagnosed from the comparison of the methylation determinations and optionally the sequence variant, or

c) the extent of methylation of the potentially aberrantly methylated DNA region, the extent of methylation in a definitely physiologically methylated DNA region of the X chromosome, and the extent of methylation as well as the sequence variant of a polymorphous DNA region of the X chromosome, which may either be physiologically or aberrantly methylated, are determined in a sample, and the presence and optionally the variant and the extent of the potential aberration are subsequently diagnosed from the comparison of the methylation determinations and the sequence variant.

Within the context of the present invention, extent of methylation is meant to denote the ratio of methylated and unmethylated alleles in a chromosome, which means that the methylated and the unmethylated DNA are simultaneously determined in a reaction. Said simultaneous determination allows a precise and quantitative assertion about the ratio of methylation to non-methylation of a specific sequence. It is, thereby, feasible to diagnose the extent and variant of the disease in question.

Within the context of the present invention, potentially aberrantly methylated DNA region is meant to denote a DNA region that is methylated or unmethylated. If this DNA region is affected by a methylation, an aberrant methylation can induce a disease.

It could be demonstrated that a quantitative methylation analysis of the two alleles of a methylated DNA region and/or the determination of the clone size via the methylation analysis of a polymorphous DNA region give an unambiguous and a clear solution as to the methylation pattern and hence the syndrome. The different genotypes of both male and female patients can thus be distinguished, because the result exhibits a specific pattern as a function of the respective genotype.

A quantitative methylation analysis which yields sound results in the determination of the clonality (clone size) is suitable for the clarification of all X-chromosomal diseases, and also those diseases which are based on a deviation from the normal number of X chromosomes (Klinefelter, Turner, Multiple X syndromes), or also those diseases whose affected cell population (clone) exhibits X aneuploidy. This is found very frequently in various tumor diseases and hematologic neoplasms. The detection and determination of the clone size/clonality of these neoplastic diseases is an important parameter in diagnosing and monitoring. In the event of autosomal diseases relating to imprinted genes, conditions are similar.

Thereby, also the extent of methylation in a definitely physiologically methylated DNA region of the X chromosome can be determined. The definitely physiologically methylated DNA region merely relates to one allele of the X chromosome, while the other allele is not methylated.

If the extent of the definitely physiologically methylated DNA region known, this additional control serves as an internal standard.

This additional analysis will be of diagnostic value if the potentially aberrantly methylated DNA region comprises deletions such that it cannot be detected irrespective of whether it comprises methylations or not. The detection of the potentially aberrantly methylated DNA region would then be missing in the result, yet the definitely physiologically methylated DNA region would be detected. From this, a deletion can be concluded. Without such an additional control, it would not be possible to tell whether the DNA region does comprise deletions, and was therefore not detected, or whether the detection has failed.

According to point b), however, another option for a possible additional determination resides in the determination of the extent of methylation as well as optionally the sequence variant of a polymorphous DNA region of the X chromosome. A syndrome is, for instance, determined by the evaluation of the potentially aberrantly methylated DNA region and the polymorphous DNA region.

In the context of the present invention, the term polymorphous DNA region serves to denote the variability of a DNA sequence and, for instance, a DNA section which comprises different alleles in a population, such as the length of a given DNA section in the event of microsatellite repeats or nucleotide and amino acid polymorphisms. The simultaneous determination of the extent of methylation of the sequence variant along with the determination of the extent of methylation of the potentially aberrantly methylated DNA region enables a perfect statement as to the variant or clone size of the respective disease.

According to point c), these three different determinations can also be carried out at one and the same time, which will result in the maximum clarification of the syndrome, since the variant of the aberrantly methylated DNA region can be precisely assigned. The method can be carried out rapidly and the result is faultless and easy to interpret. As opposed to conventional analytical procedures, this method enables the examination of both male and female patients. This analysis yields unambiguous results even in borderline cases.

The extent of methylation can be determined by various methods, e.g., by methylation-specific restriction enzymes, oligonucleotides specifically recognizing and hybridizing methylation-specifically altered DNA sections, etc. Also the sequence variant of the polymorphous DNA region can be determined in various ways such as, for instance, by PCR or by the aid of restriction enzymes.

For a rapid analysis, it is particularly advantageous if the determination of the extent of methylation as well as optionally the determination of the sequence variant are performed by a methylation-specific PCR (MS-PCR), using primers by which a methylated or unmethylated DNA region is each amplified in a methylation-specific manner. The MS-PCR technique is a simple method to distinguish between methylated sequences and unmethylated sequences and is used to diagnose patients, because methylation or non-methylation frequently is an indication for a genetically caused disease, or also gives rise to the same.

MS-PCR is based on the principle of using primers which are specific for a methylated or unmethylated sequence such that the respective ones of the primers will each hybridize with the sequence as a function of whether the latter is methylated or not. The PCR products will subsequently suggest a methylation or non-methylation. The primers may, for instance, be chosen such that the PCR product for the methylated sequence will have a certain length and the PCR product for the unmethylated sequence will have another length. The PCR products, thus, will give information about the methylation of amplified DNA sections on account of their sizes.

A common method for obtaining MS primers consists in specifically altering the methylated and/or unmethylated sequence and constructing appropriate primers that are specific for the altered sequences. An option to alter DNA in a methylation-specific manner involves treatment of the DNA with a deaminating agent such as, e.g., sodium bisulfite. Sodium bisulfite converts the unmethylated cytosine into uracil, which is replaced with thymidine in the subsequent DNA amplification. This method, thus, produces different DNA sequences, departing from originally homologous, yet differently methylated alleles. The primers that hybridize with the unmethylated sequence comprise thymidine instead of cytosine. On the other hand, the primers that are specific for the methylated sequence continue to comprise cytosine on the sites of methylated Cs.

In the instant case, it is advantageous for an unambiguous result if the primers are chosen such that the PCR product of the methylated sequence will always have another length than the PCR product of the unmethylated sequence—irrespective of the length of the repeat region.

The determination by means of MS-PCR accordingly guarantees a particularly time-saving and efficient method, requiring little DNA and involving less work. The results of PCRs are obtained simultaneously. Nor are any additional method steps like Southern blotting, hybridization methods, etc. required.

It is particularly beneficial, for instance in the event of the CGG repeat of the FMR 1 gene, if the MS-PCR primers are realized on an antisense strand or on antisense strands. The melting temperature will thereby be reduced from 95° C. to a melting temperature of 75° C., which enables the PCR to be carried out at moderate annealing temperatures, thus improving the process course. These temperature conditions are inoffensive to the reaction components and DNA polymerase.

Preferably, the MS-PCRs are carried out in duplex reactions. This means that the PCRs for the amplification of a given methylated sequence and the same given unmethylated sequence are carried out in one reaction, e.g., the PCRs for the amplification of the polymorphous methylated and unmethylated DNA regions. In this manner it is safeguarded that the reaction products obtained will be unambiguously assigned in the subsequent analysis on account of their sizes, thus rendering the result clearly interpretable.

Advantageously, the MS-PCR for the determination of the potentially aberrantly methylated DNA region and the MS-PCR for the determination of the definitely physiologically methylated DNA region are carried out in a joint multiplex reaction. Since the MS-PCRs, as a rule, concern two different genes and hence two different sequences, the MS-PCRs will not be mutually influenced. The determination of the polymorphous DNA region would be carried out in a separate duplex reaction so as to enable clear determination of the polymorphism, for instance, in order to prevent the same having the size of variable PCR products from overlaying with other PCR products in the subsequent evaluation.

In a particularly preferred manner, a method is provided, wherein the polymorphous DNA region is a DNA region with a repeat polymorphism, which is preferably connected with the potentially aberrantly methylated DNA region, whereby the length of the repeat region is determined as a sequence variant and subsequently both the presence and the extent of the potential aberration are determined.

In the context of the present invention, repeat polymorphism is meant to denote a region which comprises several repetitions of a repeat unit, i.e., a certain sequence of several nucleotides. The number of repetitions, or so-called repeat units, differs as a function of the gene or disease, the extent of the disease, as a rule, being related to the length of the repeat region. In most cases, a repeat region having a particularly unnatural length is also methylated at the same time if this DNA region is normally unmethylated. Mostly, in that case, also the, e.g., adjacent or potentially aberrantly methylated region is accordingly strongly methylated such that the determination of the potentially aberrantly methylated DNA region and the determination of the polymorphous DNA region yield complementary results and can, thus, be regarded as additional controls.

In a particularly preferred manner, the polymorphous DNA region is the CGG trinucleotide repeat region of the FMR 1 gene. The determination in this region yields a repeatable and unambiguous result. If the determination is, for instance, carried out by an MS-PCR, both the length of the repeat region and the extent of methylation can be determined in a single method step if the primers are chosen such that they encompass the whole repeat region. As already pointed out in the introductory part of the specification, the CGG trinucleotide repeat region of the FRM 1 gene is a sequence region that gives information about diseases and, in particular, the fragile X syndrome, since the length of the repeat region is characteristic of the variant of the disease.

It is particularly beneficial if the polymorphous DNA region is in the first untranslated exon of the FMR 1 gene. This is a region which is advantageous for the determination, yielding a faultless result.

Preferably, the potentially aberrantly methylated DNA region is the FMR 1 gene or a part of the same. This guarantees a facilitated procedure, since only the FMR 1 gene region is required for at least part of the analysis. It could be used for the analysis upon isolation from the remaining DNA, or the analysis could also be carried out on isolated X chromosomes.

Preferably, the potentially aberrantly methylated DNA region is the 5′-untranslated region of the FMR 1 gene, i.e., the FMR 1 promoter, or a part of the same. On the one hand, this yields reliable results and, on the other hand, the potentially aberrantly methylated DNA region is, thus, close to the CGG repeat region, which is located in the first untranslated exon. In this manner, it is not necessary to use the whole gene for the analysis, which might possibly disintegrate during deamination. If the potentially aberrantly methylated DNA region is located in the FMR 1 promoter, a shorter DNA section can be used for the assay.

In this case, it is advantageous if the definitely physiologically methylated DNA region is a DNA region that is definitely methylated on the active X chromosome. Its methylation pattern is, therefore, contrary to that of the FMR 1 gene which is methylated on the inactive X chromosome. This additional determination, thus, provides a novel and inventive method which also allows the determination of the degree of the respective disease in females in a particularly simple and unambiguous manner. To this end, the ratio of the methylated to the unmethylated DNA regions of the one gene (preferably the FMR 1 gene) is compared to the ratio of the reciprocally methylated DNA region (methylated:unmethylated) as well as the ratio of the methylated repeat polymorphism to the unmethylated repeat polymorphism (preferably of the FMR 1 gene).

Preferably, the definitely physiologically methylated DNA region is a region that comprises the XIST gene or parts of the same. The XIST gene is methylated on the active chromosome X such that it comprises a methylation pattern reciprocal to that of the FMR 1 gene. The determination by the aid of the XIST gene as an internal standard constitutes a reliable, unambiguous and simple method for detecting and evaluating a potentially aberrantly methylated DNA region on the X chromosome, in particular in females.

It is particularly favorable if the definitely physiologically methylated DNA region is a region in the XIST gene promoter. It turned out that the methylation of the promoter of the XIST gene is easy to determine and leads to reliable results.

For a particularly advantageous method it is provided that the potentially aberrantly methylated DNA region and the polymorphous DNA region are two sequences that do not overlap each other. It is, thus, ensured that, even if both DNA regions are on the FMR 1 gene, two separate, independent DNA regions are analyzed in a manner that the results of the investigations into both regions complement each other, thus enabling the premature recognition of possible confusion errors.

Preferably, two or more potentially aberrantly methylated DNA regions are simultaneously detected on the X chromosome and evaluated. It is, for instance, feasible to simultaneously determine in one reaction the syndromes relating to FraX-A, FraX-E and FraX-F, whereby an exact and reliable result will be obtained for any syndrome without the results of the different syndromes influencing one another.

A further aspect of the present invention is the use of an above-described method according to the invention for the detection and evaluation of X-chromosomal diseases. Such diseases, which have already been described above, are diseases induced by an aberrant methylation on at least one site in the X chromosome. Depending on the disease to be detected and evaluated, a specific potentially aberrantly methylated DNA region and optionally a definitely physiologically methylated DNA region of the X chromosome and/or a specific polymorphous DNA region of the X chromosome are analyzed.

In doing so, it is particularly advantageous if the method is used, in particular, for the detection and evaluation of the FraX-A, FraX-E and FraX-F syndromes, the clonality and X-aneuploidies.

In the event of the fragile X syndrome, it is particularly favorable if the FMR 1 promoter is determined as a potentially aberrantly methylated DNA region and/or the XIST gene or a part of the same is determined as a definitely physiologically methylated DNA region and/or the CGG trinucleotide region of the FMR 1 gene is determined as a polymorphous DNA region.

In the event of FraX-E, it is advantageous to determine the CpG islet at Sq28 as a potentially aberrantly methylated DNA region and/or the GCC trinucleotide region as a polymorphous DNA region and/or the XIST gene or a part of the same as a definitely physiologically methylated DNA region.

In the event of FraX-F, its is advantageous to determine the (GCCGTC)ₙ(GCC)ₘ region as a polymorphous DNA region and/or the adjacent CpG islet as a potentially aberrantly methylated DNA region and/or the XIST gene or a part of the same as a definitely physiologically methylated DNA region.

Concerning the clone size, the CGG nucleotide repeat region of the FMR1 gene is preferably determined as a polymorphous DNA region.

As already pointed out in the introductory part of the specification, normal sequences comprise a repeat region of 5-(48) 54 CGG units, premutated sequences comprise (48-) 54-200 CGG units and full mutations comprise over 200 CGG units, in the event of FraX-A.

In order to completely ascertain the syndrome in the event of the fragile X syndrome (and, of course, also with, e.g., FraX-E and FraX-F), an unambiguous and rapid distinction between the different genotypes of the fragile X syndrome can be made by the analysis according to the invention even with female patients in which methylation analyses do not yield clear results, since —concerning the repeat region—the latter is competed in a PCR, particularly at a length of more than 200 units, by a repeat of normal length. By determining the extent of methylation of the potentially aberrantly methylated DNA region, the sample can be clearly assigned to a certain genotype, since in the normal and premutation cases this DNA region is not methylated, whereas it is methylated in the event of full mutations. In male pre-/full mutation mosaics, conditions are similar to those already described for female patients. No clear diagnosis can be obtained from a conventional analysis in which merely the repeat region is determined, in particular in borderline cases, without additional method steps such as, e.g., Southern blotting. Even if Southern blotting is applied, it only is feasible to detect mosaics to a certain extent because of the limited sensitivity. By contrast, an improved assessment of the syndrome can be obtained at a reduced demand of patient DNA by the higher-sensitivity PCR methods mentioned.

If, furthermore, besides the determination of a potentially aberrantly methylated DNA region also a definitely physiologically methylated DNA region is analyzed, the latter can be employed as an internal standard. If the potentially aberrantly methylated DNA region cannot be detected on account of deletions, the definitely methylated DNA region (e.g., the XIST gene) will be detected in any event. It is, thus, guaranteed that the method did function and the potentially aberrantly methylated DNA region is possibly missing at least partially, i.e., deleted (FMR 1 gene, for instance).

In the event that the FMR 1 repeat region is determined by means of MS-PCR, the number of normal and premutation repeat units can be readily calculated from the length of the MS-PCR products. Thus, a method is provided, in which the degree of methylation and the length of the repeat regions are determined at one and the same time. The degree of the fragile X syndrome is, thus, determined in a single method step (either a single MS-PCR reaction or several MS-PCR reactions concurrently). MS-PCR is a rapid and reliable method which is cost-effective and can be carried out in any laboratory. Moreover, only a small quantity of DNA is required for this analysis. Furthermore, no additional, frequently time-consuming and expensive methods such as, for instance, immunologic or Southern blot methods need be carried out. Unlike conventional methods for determining the fragile X syndrome, which comprise PCR and an additional Southern blot in most cases, no radioactive substances are employed, which is advantageous, in particular for the laboratory personnel. In addition, a method is provided which saves cumbersome operating steps and is suitable, in particular, for routine diagnoses and screening assays. Moreover, this analysis is suitable for DNA extracted from Guthrie maps and for assays of small tissue samples taken from the body by the aid of fine needles.

If a triple MS-PCR analysis is carried out for the detection and evaluation of the fragile X syndrome, the following results will be obtained:

Normal male individuals comprise an unmethylated repeat region (as a polymorphous DNA region) of up to (48-) 54 units as well as an unmethylated FMR 1 DNA region, e.g., the promoter (as a potentially aberrantly methylated DNA region).

Male patients afflicted with a premutation exhibit the same pattern, yet with approximately (48-) 54 to 200 repeat units.

Male patients afflicted with a full mutation comprise a methylated repeat region with more than 200 units (which is usually not amplified) as well as a methylated FMR 1 promoter region (as a potentially aberrantly methylated DNA region). In full mutation/premutation borderline cases, it is decisive whether the methylated or unmethylated FMR 1 promoter region is amplified. Even if the methylated repeat region of a full mutation is as long so as not to be methylated, the full mutation can be detected because of the amplification of the methylated FMR 1 promoter region.

Mosaic full mutation/premutation: the repeat region of the allele with the full mutation is not amplified, the unmethylated repeat region of the premutation allele is amplified, comprising (48-) 54 to 200 units. The methylated FMR 1 promoter region is amplified.

On account of the random X inactivation in normal homozygous females, the ratio of the unmethylated to the methylated FMR 1 DNA region as well as of the unmethylated to the methylated XIST gene is 1:1. Since the two homologs comprise identical repeat region lengths, only one unmethylated and one methylated FMR 1 repeat region is each visible.

In heterozygous females having different repeat region lengths, the same pattern as described above is to be observed, except that two unmethylated and two methylated FMR 1 repeat regions having different lengths are to be seen.

Females afflicted by a premutation exhibit similar patterns as heterozygous females, the difference being that one methylated and one unmethylated FMR 1 repeat region of an allele have lengths of (48-) 54 to 200 units.

Females afflicted by a full mutation always comprise a methylated FMR 1 promoter region on the expanded allele irrespective of whether this is on the active or inactive chromosome. In the event of a random X inactivation, the methylation ratio of the FMR 1 DNA region promoter region is 3:1 (methylation:non-methylation), while the methylation ratio of the XIST gene is still 1:1.

Females afflicted by a full mutation and either a skewed X inactivation or a mosaic exhibit the same pattern as respective females with a random X inactivation. A skewed X inactivation and mosaics can be identified by the quantitative assessment of the methylation ratio of the FMR 1 promoter region with that of the XIST gene and the FMR 1 repeat region. The methylation ratio of the XIST gene will remain 1:1 in all cases, since every cell continues to contain an active and an inactive chromosome. The methylation ratio of the section of the further gene (XIST), thus, constitutes a standard. By contrast, the methylation ratio of the FMR 1 promoter region and that of the repeat region will shift towards non-methylation or methylation, respectively, as a function of whether the normal chromosome or that with the expanded allele comprises a skewed X inactivation. A similar pattern can be observed in mosaic women having a normal cell population and one with a full mutation.

According to another aspect, the present invention relates to a kit of the initially defined kind, which comprises primer sets for the specific amplification of the respective one of the methylated and unmethylated DNA variants of a potentially aberrantly methylated DNA region and/or a definitely physiologically methylated DNA region of the X chromosome and/or a polymorphous DNA region of the X chromosome, which may either be physiologically or aberrantly methylated. This kit serves to carry out the above-described method according to the invention.

Said kit preferably comprises, in addition to the primer sets, further substances that are necessary to carry out an MS-PCR: a substance for the conversion of the methylated or unmethylated sequence (e.g., sodium bisulfite for the conversion of unmethylated cytosine residues), the necessary enzymes, buffers, etc. In this manner, a kit is provided which only requires addition of the DNA to be assayed in order to carry out the method according to the invention. By the aid of this kit, routine diagnoses can be performed in any laboratory.

In a particularly advantageous manner, said kit can be employed for the detection and evaluation of X-chromosomal diseases, in which case it comprises primer sets for the methylation-specific amplification of a DNA region with a repeat polymorphism for the determination of the polymorphous DNA region. These primer sets serve to determine the extent of methylation and the length of the repeat polymorphism in order to assay the degree of the disease and the clonality of the same. Such chromosomal diseases include, for instance, FraX-A, FraX-E, FraX-F, X-chromosomal retardations, clonality, etc.

Preferably, the kit comprises primer sets for the amplification of a polymorphous DNA region of the CGG nucleotide repeat region in the first exon of the FMR 1 gene. Since the repeat region of the FMR 1 gene guarantees a safe and clear assertion as to the degree of disease of the fragile X syndrome or the clonicity, respectively, as already described above, a rapid and reliable analytical method can, thus, be provided by said kit.

It is, furthermore, beneficial if the kit comprises primer sets for the amplification of the XIST gene or parts of the same in order to determine the definitely physiologically methylated DNA region. As already described above, the XIST gene has a methylation pattern reciprocal to that of the FMR 1 gene. The methylation pattern in the result allows for a precise interpretation in respect to the variant of the fragile X syndrome.

It is, furthermore, advantageous if the kit comprises primer sets for the amplification of the FMR 1 gene or a part of the same in order to determine the potentially aberrantly methylated DNA region. In a favorable manner, the kit comprises primer sets for the amplification of the FMR 1 gene promoter or a part of the same. With that kit, the above-described preferred analytical method can be carried out.

In doing so, it is advantageous if the primer sets are each provided as duplex sets in a spatially separated manner. It is thereby ensured, as described above, that clear and readily interpretable results will be obtained.

It is particularly favorable if the primer sets for the amplification of the definitely physiologically methylated DNA region and the potentially aberrantly methylated DNA region are provided together in a multiplex set or a 4-plex set. Thus, the methylation-specific PCR reactions of two DNA regions are carried out simultaneously in one reaction mixture, which is why the method requires even less time. This is feasible, because the two genes (FMR 1 and XIST) comprise different sequences such that the two PCR reactions will not influence each other.

In the following, the present invention will be explained in more detail by way of examples and drawing figures to which it is, however, not to be limited, wherein:

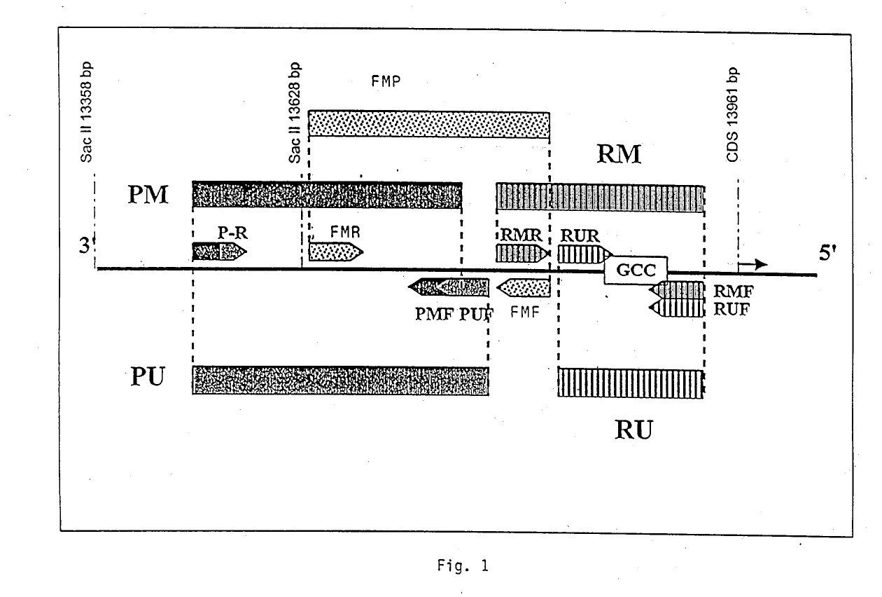

FIG. 1 illustrates the position of the primers for the FMR 1 gene;

FIG. 2 schematically illustrates the respective methylation patterns of the FMR 1 and XIST genes as well as the size and methylation of the repeat region;

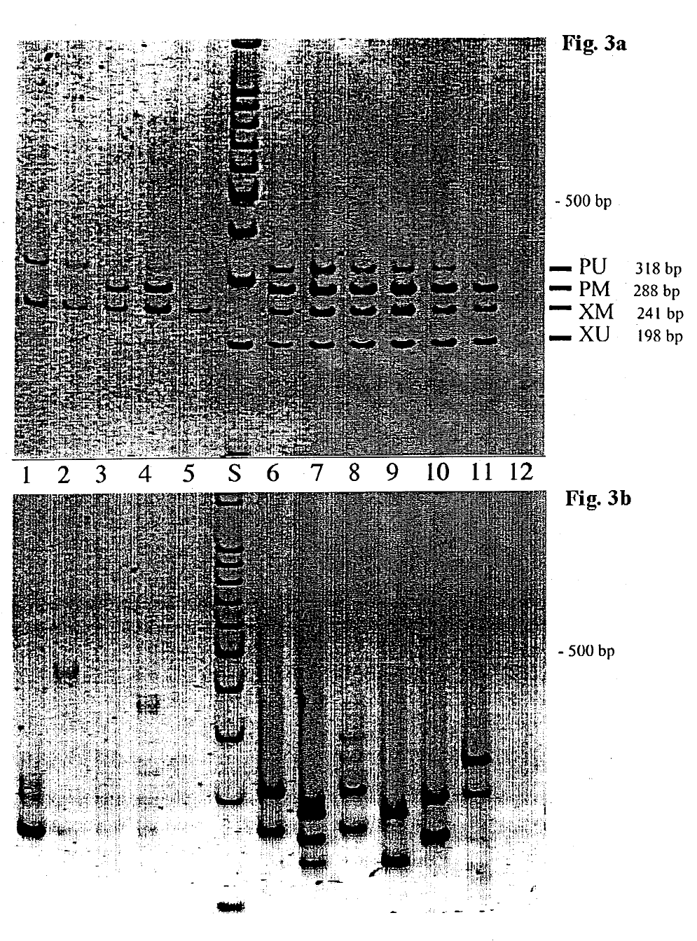

FIGS. 3a and 3b depict gel electrophoreses for the separation of the MS-PCR products;

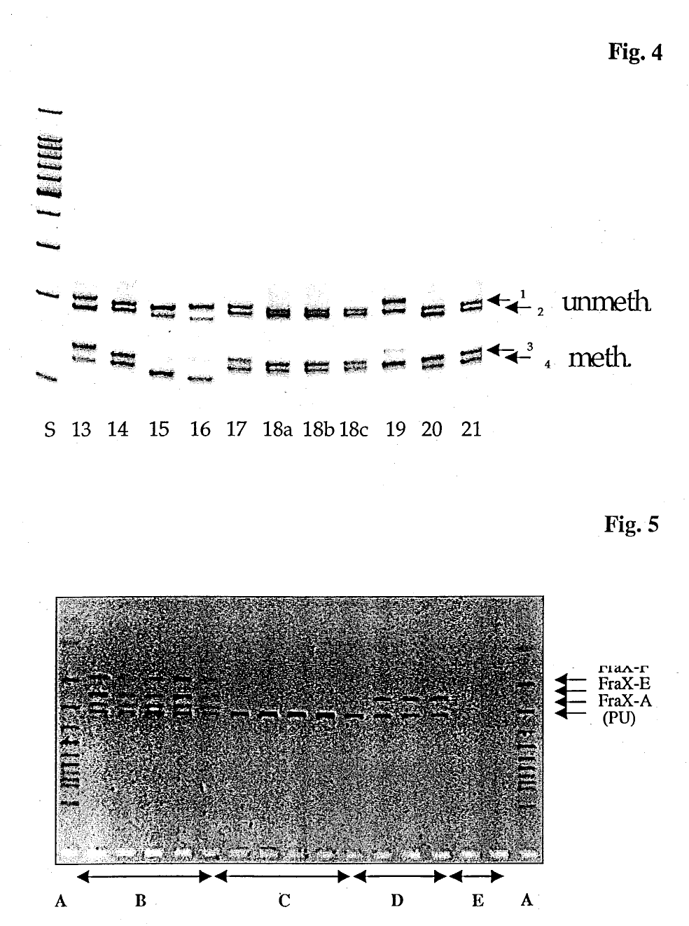

FIG. 4 shows the results of a multiplex PCR at the HUMARA locus;

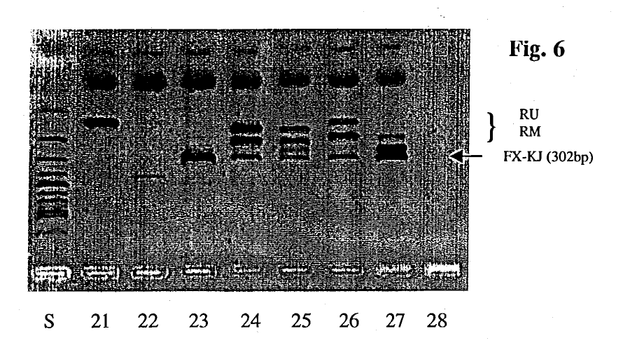

FIGS. 5 and 6 show the results of MS-PCRs.

### EXAMPLE 1

### DNA Preparation

EDTA-anticoagulated, peripheral blood samples from healthy patients as well as patients afflicted by the fragile X syndrome were stored at −20° C. in 500 μl aliquots until DNA extraction. For DNA deamination and subsequent MS-PCR analysis, DNA was extracted from 80 μl blood with DNAzol (Vienna Lab, Vienna, Austria) and resuspended in 30 μl sterile water.

0.5 μg DNA was deaminated according to the protocols of Zeschnigk et al., “A single-tube PCR test for the diagnosis of Angelman and Prader-Willi syndrome based on allelic methylation differences at the SNRPN locus”, Eur. J. Hum. Genet. 5, 94-8 (1997); “Imprinted segments in the human genome: different DNA methylation patterns in the Prader-Willi/Angelman syndrome region as determined by the genomic sequencing method”, Hum. Mol. Genet. 6, 387-95 (1997), said deamination having been carried out at 55° C. for two hours with the addition of 8 μl polyacrylic carrier to shorten DNA precipitation to 10 minutes at −20° C. The deaminated DNA was dissolved in 20 μl sterile water.

### EXAMPLE 2

### Methylation-Specific PCR

13 primers (cf. SEQ. ID. No. 1-13) were synthesized in accordance with the deaminated DNA sequence of the respective unmethylated or methylated gene regions (cf. Table 1 and FIG. 1).

Table 1

Multiplex PCR

| | | | | | | | |
|---|---|---|---|---|---|---|---|
| | | | | SEQ. | Concentration | Product | Product |
| Target | | Primer | | ID. | per reaction | designa- | length |
| sequence | Primer | designation | Sequence | No. | (pmol) | tion | (bp) |

| | | | | | | | |
|---|---|---|---|---|---|---|---|
| FMR1 | | | | | | | |
| unmethylated | forward | PUF | 5′-GTGTTTGATTGAGGTTGAATTTTTG-3′ | 1 | 5 | PU | 318 |
| Promoter | backward | P-R | 5′-ATTTAATTTCCCAC(A/G)CCACTAAATACAC-3′ | 2 | 10 | | |
| FMR1 | | | | | | | |
| methylated | forward | PMF | 5′-GTTGCGGGTGTAAATATTGAAATTACG-3′ | 3 | 5 | PM | 288 |
| Promoter | backward | P-R | 5′-ATTTAATTTCCCAC(A/G)CCACTAAATACAC-3′ | 2 | | | |
| XIST | | | | | | | |
| methylated | forward | XMF | 5′-AATTAAAGTAGGTATTCGCGGTTTCG-3′ | 4 | 8 | XM | 241 |
| Promoter | backward | XMR | 5′-TTTTTCCTTAACCCATCGAAATATCG-3′ | 5 | 8 | | |
| XIST | | | | | | | |
| unmethylated | forward | XUF | 5′-AAAAGTGGTTGTTATTTTAGATTTGTT-3′ | 6 | 8 | XU | 198 |
| Promoter | backward | XUR | 5′-CTACCTCCCAATACAACAATCACAC-3′ | 7 | 8 | | |
| FMR1 | | | | | | | |
| unmethylated | forward | RUF | 5′-TTTGAGAGGTGGGTTGTGGGTGTTTGAGG-3′ | 8 | 10 | RU | 64+3x |
| Repeat | backward | RUR | 5′-AACACCACTACCAAAAAACATACAACAACACAAC-3′ | 9 | 10 | | (CGG)N |
| FMR1 | | | | | | | |
| methylated | forward | RMF | 5′-CCGCCTCTAAACGAACGACGAACCGACGAC-3′ | 10 | 10 | RM | 120+3x |
| Repeat | backward | RMR | 5′-TTTCGAGAGGTGGGTTGCGGGCGTTCGAG-3′ | 11 | 10 | | (CGG)n |
| FMR1 | | | | | | | |
| methylated | forward | FMF | 5′-CGTCGTCGGTTCGTCGTTCGTTTAGAGGC-3′ | 12 | 10 | FP | 231bp |
| Promoter | backward | FMR | 5′-CCGACCGATTCCCAACAACGCGCATACG-3′ | 13 | 10 | | |

To a multiplex PCR mixture were added two forward primers (PUF, PMF) specific for unmethylated and methylated DNA and a mutual reverse primer (P-R) specific for the deaminated unmethylated and methylated FMR 1 promoters, as well as two forward primers (XUF, XMF) and two reverse primers (XUR, XMR) specific for the deaminated unmethylated and methylated XIST promoters. The duplex PCR mix comprises two forward primers (RUF, RMF) and two reverse primers (RUR, RMR) which are specific for the deaminated unmethylated and methylated triplet repeat regions.

An additional primer pair detects a further methylated sequence section in the FMR 1 promoter (FMF, FMR), yet cannot be combined with the other primers.

For the design of these primers, the sequence of the antisense strand of the FMR 1 gene region (Acc. No. L29074, L38501, U80460) was used as the target sequence to be amplified in order to reduce the melting temperature from Tm=95° C. to Tm=75° C.

The PCR was carried out in a reaction volume of 25 μl under oil, whereby 1 μl of the 20 μl deaminated DNA was each used from patients and normal controls. For the multiplex PCR, the amplification buffer F-511 (10 mM Tris, pH 8.8, 50 mM KCl, 1.5 mM MgCl₂, 0.1% Triton-X-100; Finnzymes Oy, Espoo, Finland) (DYN) was used; optimized buffer EXT (50 mM Tris, 15 mM NH₄Cl, 1.5 mM MgCl₂, 0.1% Triton-X-100, pH 9.0) was used for the duplex reaction with 4% DMSO and 60 mM TMAC and for the FMP amplification without amplifier (DMSO, TMAC). The dNTP concentrations were 200 μM of each nucleotide. Table 1 lists the optimum primer concentrations. The amplifications were performed on a Biometra TrioBlock (Biometra, Goettingen, Germany) and initiated with one unit of Dynazyme 501L (Finnzymes Oy, Espoo, Finland), with a first denaturation step at 95° C. for 5 minutes. The multiplex PCR profiles were 33 cycles at 95° C./30 s [Program 1], 60° C./20 s, 72° C./40 s [Program 2]. Duplex and FMP profiles included 35 cycles at 95° C./45 s, 63° C./1 min and 72° C./1 min. Finally, incubation took place at 72° C. for 7 minutes in all cases.

PCR products (5 μl) were separated on NOVEX-TBE gels (Novex, San Diego, Calif., USA) in 0.5×TBE buffer (90 mM Tris, 90 mM borate, 2 mM EDTA, pH 8.0). The bands were detected by staining with ethidium bromide (EtBr). Densitometric analyses were performed with KODAK-1D™ 2.0.2 software package (Kodak, New Haven, Conn., USA).

Repeat units in the normal and premutation ranges, but no full mutations could be amplified. The number of repeat units could be calculated from the lengths of the PCR products in normal individuals and premutation carriers.

Table 2 lists the results to be expected, wherein “−” means no PCR product, “+” means a PCR product and “2+” means two products having different lengths. In FIG. 2, the results to be expected are schematically illustrated using the same numbering as in Table 2.

TABLE 2

| | | | Duplex |
|---|---|---|---|
| | Multiplex PCR | | PCR |
| | FMR1 | XIST | FMR1 |
| Target gene sequence | Promoter | Promoter | Repeat |

| # | PCR product | PU | PM | XU | XM | RU | RM |
|---|---|---|---|---|---|---|---|
| 1 | Normal males | + | − | − | + | + | − |
| 2 | Males with premutation | + | − | − | + | + | − |
| 3 | Males with full mutation | − | + | − | + | − | − |
| 4 | Mosaic males with full muta- | − | + | − | + | + | − |
| | tion | | | | | | |
| 5 | Males with deletion | − | − | − | + | − | − |

| | | | | | |
|---|---|---|---|---|---|
| 6 | Normal females with identical | 1:1 | 1:1 | + | + |
| | repeat-lengths on both alleles | | | | |
| 7 | Normal females with different | 1:1 | 1:1 | 2+ | 2+ |
| | repeat-lengths on both alleles | | | | |
| 8 | Females with premutation | 1:1 | 1:1 | 2+ | 2+ |
| 9 | Females with full mutation | 1:3 | 1:1 | + | + |
| 10 | Females with full mutation and | (>)1:3 | 1:1 | Δ | ∇ |
| | an elevated amount of cells | | | | |
| | with active normal alleles | | | | |
| 11 | Females with full mutation and | (>)1:3 | 1:1 | ∇ | Δ |
| | an elevated amount of cells | | | | |
| | with actively affected alleles | | | | |
| 12 | Mosaic females with premuta- | (>)1:3 | 1:1 | Δ | ∇ |

| | tion | or | | or | |
|---|---|---|---|---|---|
| | | (<)1:3 | | ∇ | Δ |
| 13 | Mosaic females with full mu- | (>)1:3 | 1:1 | Δ | ∇ |

| | | |
|---|---|---|
| tation | or | or |

| (<)1:3 | ∇ | Δ |
|---|---|---|
| (1) Normal male individuals exhibit an unmethylated repeat region from up to (48−) 54 triplets as well as an unmethylated FMR 1 promoter. | | |
| (2) Male patients afflicted by a premutation exhibit the same pattern, yet with about (48−) 54 to 200 repeat units. | | |
| (3) Male patients afflicted by a full mutation exhibit in the result a methylated repeat region with more than 200 triplets (which in most cases is not amplified in the MS-PCR) as well as a methylated FMR 1 promoter. | | |
| (4) Male patients with a full mutation/premutation mosaic exhibit a methylated repeat region with more than 200 units (which is not amplified) as well as an unmethylated repeat region of (48−) 54 to 200 units and, in addition, an unmethylated and a methylated FMR 1 promoter. By the detection of the specific product of the XIST gene, a clear distinction between female and male patients is feasible in all cases. | | |
| (5) With deletions in the FMR 1 gene region to be amplified, merely the methylated PCR product of the XIST gene promoter is visible. | | |
| (6) In normal homozygous females, the ratio of unmethylated to methylated FMR 1 promoter and that of the XIST gene promoter is about 1:1. Since both homologs comprise identical repeat region lengths, only one unmethylated and one methylated FMR 1 repeat region is each visible. | | |
| (7) In heterozygous females exhibiting different repeat region lengths, the same pattern as described above can be observed, only two unmethylated and two methylated FMR 1 repeat regions having different lengths being visible. | | |
| (8) Females afflicted by a premutation exhibit a similar pattern as heterozygous females with the difference that one methylated and one unmethylated FMR 1 repeat region have a length of between (48−) 54 and 200 units each. | | |
| (9) Females afflicted by a full mutation always exhibit a methylated FMR 1 promoter on the expanded allele irrespective of whether this is located on the active or inactive chromosome. In the event of a random X inactivation, the methylation ratio of the FMR 1 promoter is 1:3 (non-methylation:methylation), while the methylation ratio of the XIST gene promoter continues to be 1:1. | | |
| (10)-(13) Females with a full mutation and either a skewed X inactivation or a mosaic exhibit the same pattern as afflicted females with a random X inactivation, yet with the methylation ratio in the FMR 1 gene being shifted in one or the other direction. A skewed X inactivation and mosaic can be identified by the semi-quantitative comparison of the methylation ratio of the FMR 1 promoter and the FMR 1 repeat region with that of the gene section of the XIST gene. The methylation ratio of the XIST | | |
| # gene remains 1:1 in all cases, since every cell continues to carry 1 active and 1 inactive chromosome. By contrast, the methylation ratio of the FMR 1 promoter and that of the repeat region shift towards non-methylation or methylation, respectively depending on whether the normal chromosome or that with the expanded allele comprises a skewed X inactivation. A similar pattern can be observed in mosaic females with a normal cell population and one with a full mutation. | | |

Thus, all possible variants can be detected to diagnose the fragile X syndrome and similar diseases such as FraX-E, FraX-F and other X-chromosomal diseases, but also the clonality and X-aneuploids.

FIGS. 3a and 3b depict gel electrophoreses of the MS-PCR products, FIG. 3a illustrating the products of the multiplex reaction (FMR 1 promoter, XIST promoter) and FIG. 3b showing the products of the duplex reaction (FMR 1 repeat expansion) with the numbering of Table 2 having been retained and n indicating the number of repeat units.

(1) Normal male patient (n=34)

(2) Male patient with a premutation (n=130)

(3) Male patient with a full mutation (n>200)

(4) Male patient with a mosaic full mutation (n>200 and n=100)

(5) Male patient with a deletion in the FMR 1 gene promoter as well as in the repeat region

(6) Normal homozygous female (n=32)

(7) Normal heterozygous female (n=23+30)

(8) Female with a premutation (n=33+66)

(9) Female with a full mutation (n>200 and n=23)

(10) Female with a full mutation and an elevated amount of cells with normal alleles due to a skewed X inactivation (n>200 and n=30; elevated amount of the RU product)

(11) Female with an elevated amount of cells with full mutations due to a skewed X inactivation (n>200 and n=46; elevated amount of the RM product)

(12) Negative control (native placenta DNA which was not deaminated prior to amplification) (s) Size standard.

Table 3 illustrates the results from a densitometric analysis of the multiplex PCR products of female patients. It shows the net intensities, the calculated ratios of unmethylated and methylated products as well as the standardized ratio based on the intergenic XIST standard ratio (XM per XU), the columns being numbered in accordance with FIGS. 1 and 3. The ratio of unmethylated and methylated XIST products specific for the inactive and active X chromose, respectively, remained stable within all female samples (mean value (XM/XU) )=1.033±0.21). In afflicted females (9 and 11), the ratios of the FMR 1 methylation based on the XIST standard ratio were outside the 95% confidence range (ratio (PU/PM)/(XM/XU)=0.32±0.06). In the event of a skewed X inactivation, which prefers the full mutation allele (col. 10), the ratio remains within the normal range and, on account of the different band intensities of the RU and RM products specific for afflicted females, the patient was diagnosed as suffering from the fragile X syndrome (col. 10, cf. also FIG. 3 below).

TABLE 3

| | Sample (net intensity) | | | | | |
|---|---|---|---|---|---|---|
| Product [bp] | 6 | 7 | 8 | 9 | 10 | 11 |
| PU [318] | 1206 | 2585 | 1122 | 1244 | 916 | 318 |
| PM [288] | 3812 | 5317 | 3863 | 6512 | 3278 | 2977 |
| XM [241] | 1177 | 2476 | 1972 | 3529 | 1854 | 1698 |
| XU [198] | 1157 | 1862 | 1700 | 3143 | 2363 | 2155 |
| Ratio | intragenic ratios | | | | | |
| (PM/PU) | 0.32 | 0.49 | 0.29 | 0.19 | 0.28 | 0.11 |
| (XM/XU) | 1.02 | 1.33 | 1.16 | 1.12 | 0.78 | 0.79 |
| | standardized intergenic ratios | | | | | |
| $\frac{( PM / PU )}{( XM / XU )}$ | 0.31 | 0.37 | 0.25 | 0.17 | 0.36 | 0.14 |

### EXAMPLE 3

### Evaluation of the Clonality at the Humara Gene Locus

In order to detect a skewed X inactivation, a PCR was carried out for the amplification of a sequence in the Humara locus (Human Androgen Receptor). Three primers were used:

| | |
|---|---|
| hum-A (um): | (SEQ ID NO: 18) |
| hum-B2 (m): | (SEQ ID NO: 19) |
| hum-C (com): | (SEQ ID NO: 20) |

wherein hum-A hybridizes with the unmethylated sequence, hum-B2 with the methylated sequence and hum-C in both cases. 25 μl hum-A [20 pmol/μl], 37.5 μl hum-B2 [20 pmol/μl], 50 μl hum-C [20 pmol/μl], 672 μl AD, 100 μl DYN and 100 μl dNTPs [2 mM] were used per batch. The PCR profiles comprised 33 cycles 95° C./20 s, 54° C./40 s, 72° C./40 s [Program 3].

Table 4 illustrates the results of a densitometric analysis of the multiplex PCR products, the result being visible in the gel illustrated in FIG. 4. Numbering of the bands in the gel of FIG. 4 corresponds with the numbering of the columns in Table 4. The net intensities are calculated from the ratios of unmethylated and methylated products, the mean value (A+A′)/2 deviating the more from 0.5 (i.e., 50% of the overall cell population) the more intensively skewing occurs. Patients 15, 16 and 19 exhibit intensive skewing.

TABLE 4

| | Genzyme# | | | | | | | | | | |
|---|---|---|---|---|---|---|---|---|---|---|---|
| Band # | 13 | 14 | 15 | 16 | 17 | 18a | 18b | 18c | 19 | 20 | 21 |
| | Net intensities | | | | | | | | | | |
| 1 | 47297 | 61104 | 74977 | 49291 | 53078 | 57523.1 | 54342.31 | 28592 | 68414.13 | 40645 | 34882 |
| 2 | 69217 | 56214.64 | 26346 | 13580 | 51270 | 67241.28 | 79051.14 | 43980 | 47394.43 | 69339 | 46600 |
| 3 | 52666 | 45023 | 1396 | 1262 | 26218 | 52003 | 48055 | 25896 | 11365 | 55649 | 38507 |
| 4 | 25810 | 53788 | 61391 | 27923 | 33167 | 42703 | 38669 | 25099 | 66379 | 35282 | 42878 |
| A = 1/(1 + 2) | 0.41 | 0.52 | 0.74 | 0.78 | 0.51 | 0.46 | 0.41 | 0.39 | 0.59 | 0.37 | 0.43 |
| B = 2/(1 + 2) | 0.59 | 0.48 | 0.26 | 0.22 | 0.49 | 0.54 | 0.59 | 0.61 | 0.41 | 0.63 | 0.57 |
| A′ = 4/(3 + 4) | 0.33 | 0.54 | 0.98 | 0.96 | 0.56 | 0.45 | 0.45 | 0.49 | 0.85 | 0.39 | 0.53 |
| B′ = 3/(3 + 4) | 0.67 | 0.46 | 0.02 | 0.04 | 0.44 | 0.55 | 0.55 | 0.51 | 0.15 | 0.61 | 0.47 |
| mean value | | | | | | | | | | | |
| (A + A′)/2 | 0.37 | 0.53 | 0.86 | 0.87 | 0.53 | 0.46 | 0.43 | 0.44 | 0.72 | 0.38 | 0.48 |
| (B + B′)/2 | 0.63 | 0.47 | 0.14 | 0.13 | 0.47 | 0.54 | 0.57 | 0.56 | 0.28 | 0.62 | 0.52 |

It is, thus, apparent that in a multiplex reaction the intensity of skewing and hence the extent of the clonality in the patient can be precisely evaluated by the method according to the invention.

### EXAMPLE 4

### Detection of FraX-A, FraX-E and FraX-F Patients in an MS-PCR

Multiplex MS-PCRs are carried out on patients in order to detect and evaluate in a single reaction the occurrence and extent of a possible aberrant methylation of the promoter regions of FraX-A, FraX-E and FraX-F. Table 5 indicates the primers employed [20 pmol/μl], the amounts of the substances used for the MS-PCRs and the product sizes to be expected, the concentration of the dNTPs being 2 mM. FIG. 5 shows the results (in the form of a gel illustration) of these multiplex MS-PCRs [Program 1], wherein A is the respective size standard, B refers to normal females, C refers to normal males, D represents male patients suffering from the FraX-E disease and E refers to native non-deaminated DNA. It is apparent that, compared to normal males, the afflicted male patients exhibit bands in the order of 248 bp, yet no FraX-F bands (191 bp) occur. All patients display the control (unmethylated promoter, PU).

TABLE 5

| | | | |
|---|---|---|---|
| μl | Primer sequences | Product size | SEQ.ID.No. |

| | | | | |
|---|---|---|---|---|
| DYN-buffer: | 100 μl | | | |
| dNTPs | 100 μl | | | |
| P-R | 24 μl | ATTTAATTTCCCAC(A/G)CCACTAAATACAC | | 2 |
| PUF | 16 μl | GTGTTTGATTGAGGTTGAATTTTTG | PU: 318 bp | 1 |
| PMF | 20 μl | GTTGCGGGTGTAAATATTGAAATTACG | PM: 288 bp | 3 |
| FRAX E-A | 12 μl | TTCGTCGTCGTTGTCGTCGTC | | 14 |
| FRAX E-B | 12 μl | AACTAAAAATATCCGAACCGCATCGAC | FRAX E AB: 248 bp | 15 |
| FRAX F-A | 16 μl | AGTTCGTAGCGCGGATTTTCG | | 16 |
| FRAX F-B | 16 μl | AACGTAAACGCGACTAACGCTAACG | FRAX F AB: 191 bp | 17 |
| AD | 438 μl | | | |

In the case of females, the net intensities of the bands in the gel are measured, whereby conclusions as to the extent of the disease can be drawn from the mutual intensities of the individual bands. If, for instance, one of the bands specific for the methylated promoter (PM, FraX-E AB, FraX-F AB) is amplified relative to the other bands at a ratio of 3:2, this is a sign of a disease. In the instant gel of FIG. 5, all females show ratios of 2:2, from which it can be taken that these females are healthy (suffering neither from FraX-A or FraX-E nor from FraX-F).

### EXAMPLE 5

### Combination of a Duplex PCR with a Potentially Methylated DNA Region

A PCR [Program 2] is carried out for the amplification of the polymorphous gene region (in the instant case the FX repeat (RU, RM)) along with the potentially aberrantly methylated gene region (in the example, the methylated FMR 1 promoter (FX-JK)). From the evaluation of the band patterns and the band intensities as illustrated in FIG. 6, a disease can be concluded and, if desired, the extent of the affection can be determined.

Table 6 indicates the different constellations, “XY” representing male and “XX” representing female patients.

TABLE 6

| | | | full | | | full |
|---|---|---|---|---|---|---|
| PCR product | normal XY | premut. XY | mut. XY | normal XX | premut. XX | mut. XX |
| methylated promoter: FX-JK | — | — | 1 | 2 | 2 | 3 |
| methylated repeat: RM | ‘3 | — | −(1) | 2 or 2x1 | 1 (2x1) | 1 (2x1) |
| unmethylated repeat: RU | 1 | 1 | — | 2 or 2x1 | 1 (2x1) | 1 |

Table 7 indicates the primers used and the PCR products to be expected.

TABLE 7

Primer Concentrations (20 pmol/μl)

| | | |
|---|---|---|
| Primer | 1 | Prod. size; |

| | | |
|---|---|---|
| RUF | 30 | |
| RUR | 30 | RU: |
| | | 64 + 3x(CGG)n |
| RMF | 20 | |
| RMR | 20 | RM: |
| | | 120 + 3x(CGG)n |
| FX-K | 20 | |
| FX-J | 20 | FX-JK: |
| | | 302bp |
| TMAC (1,5M) | 40 | |
| DMSO | 40 | |
| EXT | 100 | |
| dNTPs | 100 | |
| AD | 468 | |

(SEQ ID NO: 21)

| | |
|---|---|
| FX-K | 5′-GGAAGTGAAATCGAAACGGAGTTGAGC-3′ |

(SEQ ID NO: 22)

| | |
|---|---|
| FX-J | 5′-AACGTTCTAACCCTCGCGAAACAATACG-3′ |

FIG. 6 depicts the separation of the PCR products, S being the standard, 21 a normal male, 22 a male patient with premutation, 23 a male patient with full mutation, 24 a normal female (homozygous repeat), 25 a normal female (heterozygous repeat), 26 a female patient with premutation, 27 a female patient with full mutation, 28 native non-deaminated DNA as a negative control.

In a normal male only the unmethylated repeat is amplified. In male a patient afflicted by a permutation also the unmethylated expanded repeat is amplified.

In a male patient afflicted by a full mutation the methylated promoter is amplified, and optionally also the methylated repeat (as already indicated, the repeat is not amplified if it exceeds a certain size).

Normal females exhibit an intensity ratio of 2:2:2 (methylated promoter:methylated repeat:unmethylated repeat), homozygous females having two identical alleles (identical repeat number), heterozygous females, however, having different repeat lengths such that two different alleles with different repeat lengths (2×1) are to be observed.

Females with permutations exhibit a ratio of 2:1:1 (the expanded repeat cannot be amplified from a certain size; however, if it is amplified, the normal and expanded repeats are to be observed).

Females with full mutations exhibit a ratio of 3:1:1 (so far, an expanded methylated full mutation repeat could not be amplified).

It is apparent that this embodiment of the method according to the invention guarantees an exact evaluation of the syndrome both in male and in female patients.

1. A method for detecting and evaluating a potentially aberrantly methylated DNS region on the X chromosome, or the clonality, characterized in that
a) either the extent of methylation of the potentially aberrantly methylated DNA region and/or the extent of methylation in a definitely physiologically methylated DNA region of the X chromosome is determined in a sample and the presence and optionally the variant and the extent of the potential aberration are subsequently diagnosed from the comparison of the methylation determinations, or
b) the extent of methylation of the potentially aberrantly methylated DNA region and/or the extent of methylation, as well as optionally the sequence variant, of a polymorphous DNA region of the X chromosome, which may either be physiologically or aberrantly methylated, is determined in a sample, and the presence and optionally the variant and the extent of the potential aberration, or the clonality, are subsequently diagnosed from the comparison of the methylation determinations and optionally the sequence variant, or
c) the extent of methylation of the potentially aberrantly methylated DNA region, the extent of methylation in a definitely physiologically methylated DNA region of the X chromosome, and the extent of methylation as well as the sequence variant of a polymorphous DNA region of the X chromosome, which may either be physiologically or aberrantly methylated, are determined in a sample, and the presence and optionally the variant and the extent of the potential aberration are subsequently diagnosed from the comparison of the methylation determinations and the sequence variant.

2. A method according to claim 1, characterized in that the determination of the extent of methylation as well as optionally the determination of the sequence variant are carried out by a methylation-specific PCR (MS-PCR), using primers by which a methylated or unmethylated DNA region is each amplified in a methylation-specific manner.

3. A method according to claim 2, characterized in that the MS-PCRs are carried out on an antisense strand or on antisense strands.

4. A method according to claim 2 or 3, characterized in that the MS-PCRs are carried out in duplex reactions.

5. A method according to claim 2 or 3, characterized in that the MS-PCR for the determination of the potentially aberrantly methylated DNA region and the MS-PCR for the determination of the definitely physiologically methylated DNA region are carried out in a joint multiplex reaction.

6. A method according to any one of claims 1 to 5, characterized in that the polymorphous DNA region is a DNA region with a repeat polymorphism, which is preferably connected with the potentially aberrantly methylated DNA region, whereby the length of the repeat region is determined as a sequence variant and subsequently both the presence and the extent of the potential aberration are determined.

7. A method according to any one of claims 1 to 6, characterized in that the polymorphous DNA region is the CGG trinucleotide repeat region of the FMR 1 gene.

8. A method according to claim 7, characterized in that the polymorphous DNA region is located in the first untranslated exon of the FMR 1 gene.

9. A method according to any one of claims 1 to 8, characterized in that the potentially aberrantly methylated DNA region is the FMR 1 gene or a part of the same.

10. A method according to any one of claims 1 to 9, characterized in that the potentially aberrantly methylated DNA region is the 5′-untranslated region of the FMR 1 gene, i.e., the FMR 1 promoter, or a part of the same.

11. A method according to any one of claims 1 to 10, characterized in that the definitely physiologically methylated DNA region is a DNA region definitely methylated on the active X chromosome.

12. A method according to any one of claims 1 to 11, characterized in that the definitely physiologically methylated DNA region is a region that comprises the XIST gene or parts of the same.

13. A method according to any one of claims 1 to 12, characterized in that the definitely physiologically methylated DNA region is a region in the XIST gene promoter.

14. A method according to any one of claims 1 to 13, characterized in that the potentially aberrantly methylated DNA region and the polymorphous DNA region are two sequences that do not overlap each other.

15. A method according to any one of claims 1 to 14, characterized in that two or more potentially aberrantly methylated DNA regions are simultaneously detected on the X chromosome and evaluated.

16. The use of a method according to any one of claims 1 to 15 for the detection and evaluation of X-chromosomal diseases.

17. The use of a method according to claim 16, characterized in that it is used, in particular, for the detection and evaluation of the FraX-A, FraX-E and FraX-F syndromes, the clonality and X-aneuploidies.

18. A kit for the detection and evaluation of a potentially aberrantly methylated DNA region on the X chromosome by a method according to any one of claims 2 to 15, characterized in that it comprises primer sets for the specific amplification of the respective one of the methylated and unmethylated DNA variants of a potentially aberrantly methylated DNA region and/or a definitely physiologically methylated DNA region of the X chromosome and/or a polymorphous DNA region of the X chromosome, which may either be physiologically or aberrantly methylated.

19. A kit according to claim 18 for the detection and evaluation of X-chromosomal diseases, characterized in that it comprises primer sets for the methylation-specific amplification of a DNA region with a repeat polymorphism for the determination of the polymorphous DNA region.

20. A kit according to claim 18 or 19, characterized in that it comprises primer sets for the amplification of a polymorphous DNA region of the CGG nucleotide repeat region in the first exon of the FMR 1 gene.

21. A kit according to any one of claims 18 to 20, characterized in that it comprises primer sets for the amplification of the XIST gene or parts of the same in order to determine the definitely physiologically methylated DNA region.

22. A kit according to any one of claims 18 to 21, characterized in that it comprises primer sets for the amplification of the FMR 1 gene or a part of the same in order to determine the potentially aberrantly methylated DNA region.

23. A kit according to claim 22, characterized in that it comprises primer sets for the amplification of the FMR 1 gene promoter or a part of the same.

24. A kit according to any one of claims 18 to 23, characterized in that the primer sets are each provided as duplex sets in a spatially separated manner.

25. A kit according to any one of claims 18 to 23, characterized in that the primer sets for the amplification of the definitely physiologically methylated DNA region and the potentially aberrantly methylated DNA region are provided together in a multiplex set or a 4-plex set.
